(19) United States
(12) Patent Application Publication
(10) Pub. No.: US 2023/0027128 A1
(43) Pub. Date: Jan. 26, 2023

(54) Endotracheal Tube and Endotracheal Sleeve and Methods of Introducing Endotracheal Tube to a Patient

(71) Applicant: Holly Lashmet, Bartley, NE (US)

(72) Inventor: Holly Lashmet, Bartley, NE (US)

(21) Appl. No.: 17/862,493

(22) Filed: Jul. 12, 2022

Related U.S. Application Data
(60) Provisional application No. 63/221,258, filed on Jul. 13, 2021.

(51) Int. Cl. A61M 16/04

(52) U.S. Cl. CPC A61M 16/0488; A61M 16/0434

(57) ABSTRACT

An endotracheal safety device comprising an endotracheal tube having a proximal end and a distal end, the endotracheal tube defining a lumen between the proximal end and the distal end, an endotracheal sleeve assembly having a proximal end and a distal end, the endotracheal sleeve assembly defining a lumen between the proximal end and the distal end, the endotracheal sleeve assembly configured to be movable over the endotracheal tube, and an adapter having a proximal end and a distal end, the distal end of the adapter configured to be received by the proximal end of the endotracheal sleeve assembly.

### CROSS REFERENCE TO RELATED APPLICATIONS

This application claims priority to U.S. Provisional Application No. 63/221,258 filed Jul. 13, 2021, the disclosure of which is hereby incorporated by reference in its entirety.

### BACKGROUND OF THE INVENTION

### Field of the Invention

This invention is directed to an endotracheal safety device and endotracheal tube and method of safely introducing the endotracheal safety device that allows prevention of over insertion of the endotracheal tube, easier insertion of the endotracheal tube, a clear visualization of depth markings of the endotracheal tube, and reliably securing the device in place.

### Description of Related Art

The current endotracheal tubes currently are either taped to the patient directly or the endotracheal tube is crimped for securement to the patient. Once in place, current endotracheal tubes holders do not allow for easy adjustment of the endotracheal tube. Also, current endotracheal tubes holders do not allow for easy visualization of how deep the endotracheal tube is inserted once in place. Further, current endotracheal tubes are too flexible and a stylet may be used to successfully intubate a patient and the endotracheal tube becomes easily disconnected from a breathing circuit, which causes loss of ventilation and oxygenation for the patient. The current invention improves upon the deficiencies of current endotracheal tubes and endotracheal tube holders.

### SUMMARY OF THE INVENTION

The current invention is directed to an endotracheal safety device comprising an endotracheal tube having a proximal end and a distal end, the endotracheal tube defining a lumen between the proximal end and the distal end; an endotracheal sleeve assembly having a proximal end and a distal end, the endotracheal sleeve assembly defining a lumen between the proximal end and the distal end, the endotracheal sleeve assembly configured to be movable over the endotracheal tube; and an adapter having a proximal end and a distal end, the distal end of the adapter configured to be received by the proximal end of the endotracheal sleeve assembly.

The endotracheal safety device may be bent at its lower portion without the use of an intubating stylet. The endotracheal safety device may further comprise a cuff, wherein the cuff is secured on an outer surface of the endotracheal tube at a lower portion of the endotracheal tube, wherein the cuff is inflatable. The endotracheal safety device's distal end may be tapered. The proximal end of the endotracheal tube and the distal end of the adapter may be configured to be rotationally secured with respect to each other. The endotracheal tube may have at least one marking on the outer surface. The endotracheal sleeve assembly may comprise a clamp, wherein the clamp is configured to secure the endotracheal sleeve assembly in place such that the endotracheal sleeve assembly is not movable with respect to the endotracheal tube.

The endotracheal sleeve assembly may further comprise a flange wherein the flange defines at least one hole. The endotracheal sleeve assembly may further comprise a strap wherein the strap may be attached to the flange. The strap may be attached to the at least one hole of the flange. The endotracheal sleeve assembly may further comprise a cuff inflation port, wherein the cuff inflation port is in fluid communication with the cuff such that the cuff may be inflated by introducing air to the cuff inflation port. The endotracheal safety device may comprise at least one slit and a coupler, wherein when the coupler has a first position and a second position over an outer surface of the endotracheal sleeve assembly, wherein the endotracheal sleeve assembly is configured to move with respect to the endotracheal tube when the coupler is in its first position, and wherein the endotracheal sleeve assembly is secured to the endotracheal tube when the coupler is in the second position.

An endotracheal sleeve assembly may comprise a proximal end and a distal end, the endotracheal sleeve assembly defining a lumen between the proximal end and the distal end, the endotracheal sleeve assembly configured to be movable over the endotracheal tube. The endotracheal sleeve assembly may comprise a clamp, wherein the clamp is configured to secure the sleeve assembly in place such that the endotracheal sleeve assembly is not movable with respect to an endotracheal tube. The endotracheal sleeve may further comprise at least one stop. The endotracheal sleeve assembly may further comprise a flange, wherein the flange defines at least one hole. The endotracheal sleeve assembly may further comprise a strap, wherein the strap is attached to the sleeve. The strap may be attached to the at least one hole of the flange. The endotracheal sleeve assembly may comprise at least one slit and a coupler, wherein when the coupler has a first position and a second position over an outer surface of the endotracheal sleeve assembly, wherein the endotracheal sleeve assembly is configured to move with respect to an endotracheal tube when the coupler is in its first position, and wherein the endotracheal sleeve assembly is secured to the endotracheal tube when the coupler is in the second position.

A method of introducing an endotracheal tube into a patient may comprise inserting a distal end of the endotracheal tube into the patient; sliding an endotracheal sleeve assembly over the endotracheal tube to a desired length of the endotracheal tube; and fastening the endotracheal sleeve assembly with respect to the endotracheal tube such that the endotracheal sleeve assembly does not slide with respect to the endotracheal tube. The method may further comprising providing a strap; attaching the strap to a flange of the endotracheal sleeve assembly; wrapping the strap around the patient's head; and securing the strap to an opposing side of the flange. The method may further comprise inflating a cuff of the endotracheal tube by introducing air to a cuff inflation port of the endotracheal tube and straightening the endotracheal tube by exposing the endotracheal tube to the patient's body temperature. The method may further comprise fastening a distal end of an adapter to a proximal end of the endotracheal tube.

Other exemplary embodiments or aspects are set forth in the following numbered clauses:

Clause 1: An endotracheal safety device comprising: an endotracheal tube having a proximal end and a distal end, the endotracheal tube defining a lumen between the proximal end and the distal end, an endotracheal sleeve assembly having a proximal end and a distal end, the endotracheal sleeve assembly defining a lumen between the proximal end and the distal end, the endotracheal sleeve assembly configured to be movable over the endotracheal tube; and an adapter having a proximal end and a distal end, the distal end of the adapter configured to be received by the proximal end of the endotracheal sleeve assembly.

Clause 2: The device of clause 1, wherein a lower portion of the endotracheal tube is bent.

Clause 3: The device of clause 1 or 2 further comprising a cuff, wherein the cuff is secured on an outer surface of the endotracheal tube at a lower portion of the endotracheal tube, wherein the cuff is inflatable.

Clause 4: The device of any of clause 1-3, wherein the distal end of the endotracheal tube is tapered.

Clause 5: The device of any of clauses 1-4, wherein the proximal end of the endotracheal tube and the distal end of the adapter are rotationally secured.

Clause 6: The device of any of clauses 1-5, wherein the endotracheal tube has at least one marking on the outer surface.

Clause 7: The device of any of clauses 1-6, wherein the endotracheal sleeve assembly comprises a clamp, wherein the clamp is configured to secure the endotracheal sleeve assembly in place such that the endotracheal sleeve assembly is not movable with respect to the endotracheal tube.

Clause 8: The device of any of clauses 1-7, wherein the endotracheal sleeve assembly further comprises a flange, wherein the flange defines at least one hole.

Clause 9: The device of any of clauses 1-8, wherein the endotracheal sleeve assembly further comprises a strap, wherein the strap is attached to the endotracheal sleeve assembly.

Clause 10: The device of any of clauses 1-9 further comprising a cuff inflation port, wherein the cuff inflation port is in fluid communication with the cuff such that cuff may be inflated by introducing air to the cuff inflation port.

Clause 11: The device of any of clauses 1-10, wherein the endotracheal sleeve assembly comprises at least one slit and a coupler, wherein when the coupler has a first position and a second position over an outer surface of the endotracheal sleeve assembly, wherein the endotracheal sleeve assembly is configured to move with respect to the endotracheal tube when the coupler is in its first position, and wherein the endotracheal sleeve assembly is secured to the endotracheal tube when the coupler is in the second position.

Clause 12: An endotracheal sleeve assembly comprising a proximal end and a distal end, the endotracheal sleeve assembly defining a lumen between the proximal end and the distal end, the endotracheal sleeve assembly configured to be movable over the endotracheal tube.

Clause 13: The endotracheal sleeve of clause 12, wherein the endotracheal sleeve assembly comprises a clamp, wherein the clamp is configured to secure the endotracheal sleeve assembly in place such that the endotracheal sleeve assembly is not movable with respect to an endotracheal tube, wherein the endotracheal sleeve assembly comprises at least one stop.

Clause 14: The endotracheal sleeve of clause 12 or 13, wherein the endotracheal sleeve assembly further comprises a flange, wherein the flange defines at least one hole.

Clause 15: The endotracheal sleeve of any of clauses 12-14, wherein the endotracheal sleeve assembly further comprises a strap, wherein the strap is attached to the sleeve.

Clause 16: The endotracheal sleeve of any of clauses 12-15, wherein the endotracheal sleeve assembly comprises at least one slit and a coupler, wherein when the coupler has a first position and a second position over an outer surface of the endotracheal sleeve assembly, wherein the endotracheal sleeve assembly is configured to move with respect to an endotracheal tube when the coupler is in its first position, and wherein the sleeve assembly is secured to the endotracheal tube when the coupler is in the second position.

Clause 17: A method of introducing an endotracheal tube into a patient comprising: inserting a distal end of the endotracheal tube into the patient; sliding an endotracheal sleeve assembly over the endotracheal tube to a desired length of the endotracheal tube; and fastening the endotracheal sleeve assembly with respect to the endotracheal tube such that the endotracheal sleeve assembly does not slide with respect to the endotracheal tube.

Clause 18: The method of clause 17 further comprising: providing a strap; attaching the strap to a flange of the endotracheal sleeve assembly; wrapping the strap around the patient's head; and securing the strap to an opposing side of the flange.

Clause 19: The method of clause 17 or 18 further comprising inflating a cuff of the endotracheal tube by introducing air to a cuff inflation port of the endotracheal tube and straightening the endotracheal tube by exposing the endotracheal tube to the patient's body temperature.

Clause 20: The method of any of clauses 17-19 further comprising fastening a distal end of an adapter to a proximal end of the endotracheal tube.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings.

FIG. 1 is a perspective view of an endotracheal safety device according to a non-limiting embodiment or aspect described herein.

FIG. 2 is a front view of the endotracheal safety device of FIG. 1

FIG. 3 is a perspective view of an endotracheal sleeve assembly according to a non-limiting embodiment or aspect described herein.

FIG. 4 is a front view of the endotracheal sleeve assembly of FIG. 3.

FIG. 5 is an illustration of the endotracheal safety device of FIG. 1 before assembled.

FIG. 6 is a perspective view of the endotracheal safety device secured to a patient.

FIG. 7 is a cross-sectional view of the endotracheal safety device and the patient of FIG. 6.

FIG. 8 is a perspective view of an endotracheal safety device and an endotracheal sleeve assembly in packaging.

FIG. 9 is a perspective view of an endotracheal safety device and an endotracheal sleeve assembly according to a non-limiting embodiment or aspect described herein.

FIG. 10 is side view of two endotracheal tubes showing changes in the bend and position of a cuff.

FIG. 11 is a side view of the endotracheal sleeve assembly in a pre-secured position.

FIG. 12 is a side view of the endotracheal sleeve assembly in a secured position.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

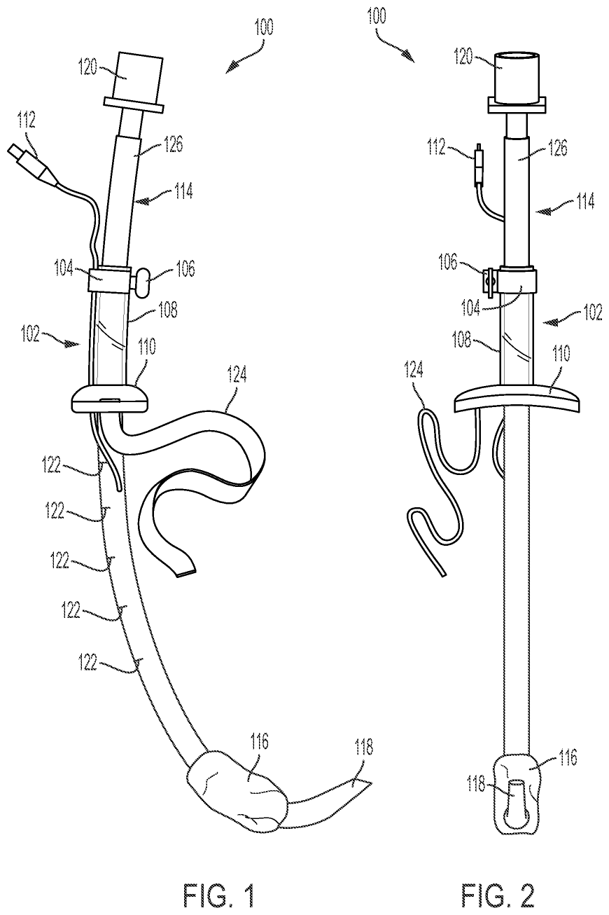

Reference is made to FIGS. 1-2, which show an endotracheal safety device 100. The endotracheal safety device 100 may include an endotracheal tube 114, an adapter 120, and an endotracheal sleeve assembly 102. The endotracheal sleeve assembly 102 may include a clamp 104, a tightening device 106, a flange 110, and a strap 124. The endotracheal tube 114 may have a proximal end 126 and a distal end 118. The endotracheal tube 114 may have at least one marking 112 on the outer surface of the endotracheal tube 114. The endotracheal tube 114 may have a cuff inflation port 112 for inflating the cuff 116 and at least one marking 122.

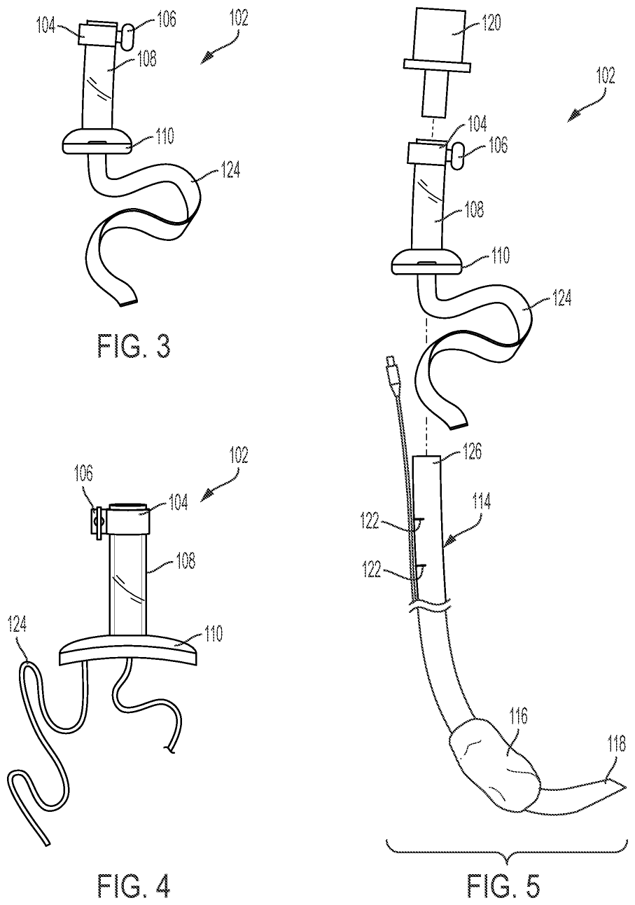

With continued reference to FIGS. 1-2 and further reference to FIGS. 3-4, endotracheal sleeve assembly 102 may have a proximal end and a distal end. The proximal end may define at least one stop. The endotracheal sleeve assembly 102 may define a lumen between the proximal end and a distal end such that the lumen of the endotracheal sleeve assembly 102 may fit over the endotracheal tube 114. The endotracheal sleeve assembly 102 is configured such that the endotracheal sleeve assembly 102 is slidably movable over the endotracheal tube 114. The endotracheal sleeve assembly 102 may include a clamp 104 for securing the endotracheal sleeve assembly 102 onto the endotracheal tube 114 such that the endotracheal sleeve assembly 102 is not slidably movable. The clamp 104 may be tightened via a tightening device 106, such as a thumbscrew. The endotracheal sleeve assembly 102 may be tightened and secured to the endotracheal tube 114 via other conventional means. The endotracheal sleeve assembly 102 may be made of any suitable material. For example, the endotracheal sleeve assembly 102 may be made of rigid, clear plastic. The clear plastic allows the markings 122 to be visible when the endotracheal sleeve assembly 102 is placed over the endotracheal tube 114. The endotracheal sleeve assembly 102 may vary in length. For example, the sleeve may be 1-5 inches long, 1-2.5 inches long, 1-2 inches long, or any suitable length.

With continued reference to FIGS. 1-2 and further reference to FIG. 3-4, the endotracheal sleeve assembly 102 may include a flange 110. The flange 110 may be integral with the endotracheal sleeve assembly 102 or removably attached to the endotracheal sleeve assembly 102. The flange 110 may have a concave shape so as to ergonomically fit over a patient's mouth. The endotracheal sleeve assembly 102 may include a strap 124. The strap may be attached to the flange 110 or the endotracheal sleeve assembly 102. The strap may 124 may be secured around a patient's head to hold the endotracheal safety device 100 in place once the endotracheal tube 114 is inserted into the patient. The strap 124 may be made of any suitable material, such as Velcro. Use of the strap 124 eliminates the need to use tapes to secure the endotracheal safety device 100 to a patient.

Now with reference to FIG. 5, the endotracheal safety device 100 may be assembled by combining the endotracheal tube 114, the endotracheal sleeve assembly 102, and the adapter 120. The lumen of the endotracheal sleeve assembly 102 goes over the outer surface of the endotracheal tube 114. The adapter 120 may have a distal end having a circumference smaller than that of the proximal end of the endotracheal tube 114 such that the adapter 120 may be received by the endotracheal tube 114. Alternatively, the adapter 120 may have a distal end having a circumference bigger than that of the proximal end of the endotracheal tube 114 such that the endotracheal tube 114 is received by the adapter 120. The adapter 120 and the endotracheal tube 114 may be secured together by friction fit, for example, the endotracheal tube 114 and the adapter 120 may have threads to allow a screw fit. Alternatively, the adapter120 and the endotracheal tube 114 may be held in place by any other traditional means. Furthermore, the adapter 120 and the endotracheal tube 114 may be integrally connected to each other. The adapter 120 with an integral connection prevents the endotracheal tube 114 from being easily disconnected from the breathing circuit, thereby preventing loss of oxygenation and ventilation.

With continued reference to FIG. 5, the endotracheal tube 114 may have a proximal end 126 and a distal end 118. The proximal end 126 may be configured to receive the adapter 120. The inner surface of the lumen of the endotracheal tube 114 at its proximal end may comprise threads that can provide a screw friction fit with the adapter 120. The distal end 118 of the endotracheal tube 114 may be tapered, such that the circumference at the distal end 118 is smaller than the rest of the endotracheal tube. 114. For example, the distal end 118 may be less than 75%, less than 50%, less than 25%, one half centimeter smaller, or any other suitable size smaller than the size of the rest of the endotracheal tube 114. The distal end 118 may be beveled. The distal end 118 may also be bent. For example, the distal end 118 may be bent at an angle between 0-50 degrees, 20-40 degrees, and 25-35 degrees. Furthermore, the distal side of the endotracheal tube 114 may be bent at a substantially 90 degree angle. For example, the distal side of the endotracheal tube 114 may be bent at an angle between 60 degrees and 90 degrees, 65 degrees and 85 degrees, and 70 degrees and 80 degrees. Such bend in the endotracheal tube 114 allows for easier placement of the endotracheal tube 114 into the trachea of a patient without the use of an intubating stylet. The endotracheal tube 114 may straighten itself out once the endotracheal tube 114 is inserted into a patient and warms up to the patient's body temperature. The endotracheal tube 114 may include a cuff 116. The cuff 116 may be in fluid communication with the cuff inflation port 112 such that air introduced into the cuff inflation port 112 may in turn inflate the cuff 116. For example, the cuff 116 may be inflated once the cuff 116 is introduced into a patient beyond the vocal cords. The endotracheal tube 114 may have one or more markings 122. The markings 122 may indicate how far the endotracheal tube 114 has been inserted into a patient, as well as mark where on the endotracheal tube 114 the endotracheal sleeve assembly 102 is secured. For example, the markings 112 may be any units of measurement, such as centimeters or units, or any indication of desired length of insertion.

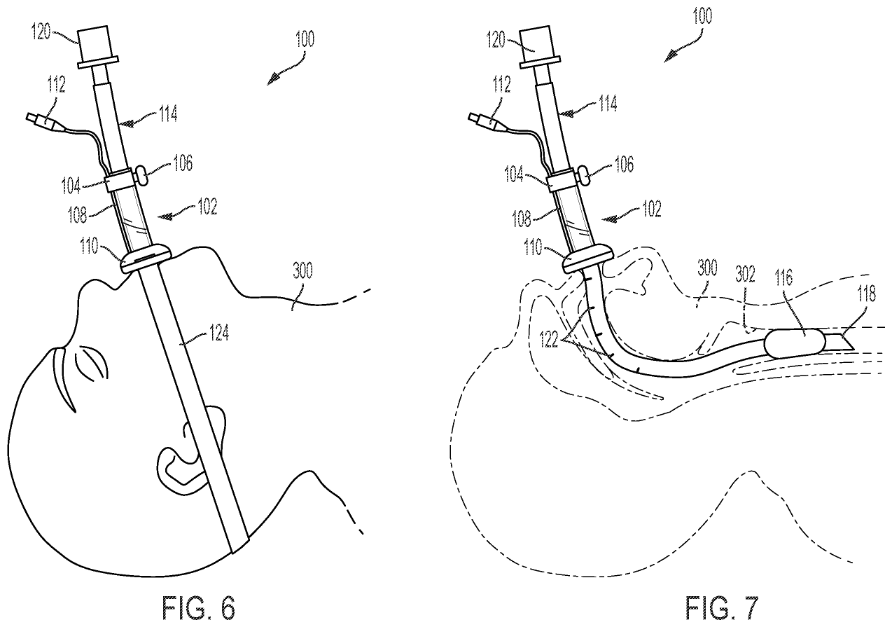

With reference to FIGS. 6 and 7, an end user may prepare the endotracheal safety device 100. For example, the end user may place the endotracheal sleeve assembly 102 over the endotracheal tube 114 such that the endotracheal sleeve assembly 102 is easily movable over the endotracheal tube 114. The end user may connect the adapter 120 to the endotracheal tube 114. The end user may slide the distal end 118 of the endotracheal tube 114, with the cuff 116 deflates, through the vocal cords. After putting together the endotracheal safety device 100, the end use may slide the cuff 116 from the distal end 118 to where the bend begins at the distal side of the endotracheal tube 114. The end user may insert the endotracheal safety device 100 into the trachea 302 of a patient 300 by inserting the distal end 118 of the endotracheal tube 114. For example, the endotracheal safety device 100 may be inserted at 21 cm, or any suitable distance, from the patient's lip. The end user may determine the how much of the endotracheal tube 114 has been inserted by examining the one or more markings 122, as well as auscultating the lungs and checking for positive ETCO2. Once a desired length of the endotracheal tube 114 is inserted, which the end user can determine by looking at the markings 122, the end user may slidably move the endotracheal sleeve assembly 102 towards the mouth of the patient 300 to such that the flange 110 of the endotracheal sleeve assembly 102 is in contact with the mouth of the patient 300. Once the endotracheal sleeve assembly 102 is in its desired position, the end user may secure the endotracheal sleeve assembly 102 in place with respect to the endotracheal tube 114 by tightening the clamp 104 via the tightening device 106. The end user then may inflate the cuff 116 by introducing air into the cuff inflation port 112. The end user may secure the endotracheal safety device 100 on the patient 300 by placing the strap 124 around the patient's head and securing both ends of the strap 124 be on the flange 106.

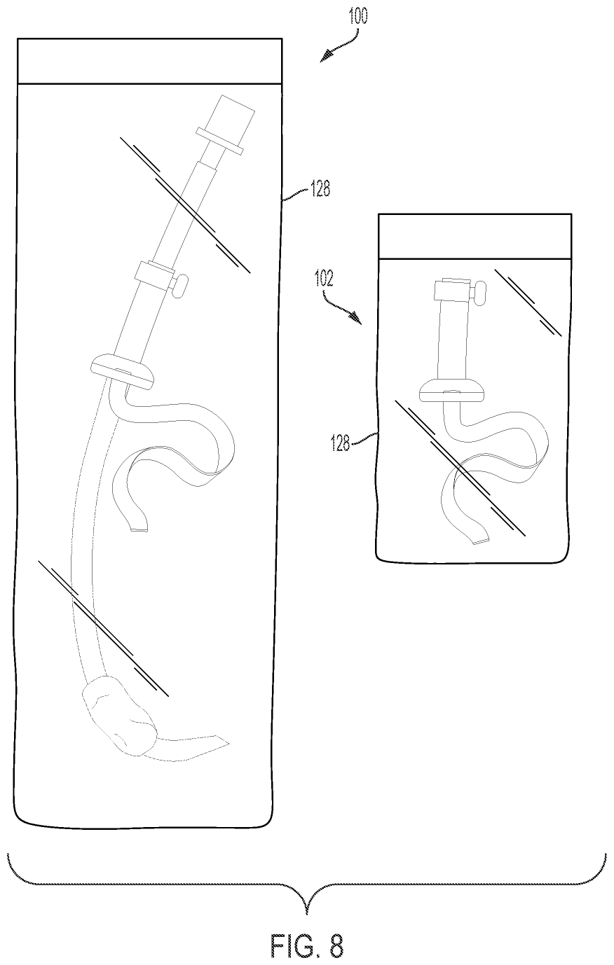

With reference to FIG. 8, the endotracheal safety device 100 may be placed in sterile packaging 128 before use for storage purposes. The endotracheal safety device 100 may be packaged such that the endotracheal sleeve assembly 102, the endotracheal tube 114, and the adapter 120 are contained in one packaging 128, as shown on the left. Alternatively, separate components of the endotracheal safety device 100 may be packaged. For example, only the endotracheal sleeve assembly 102 and its related components may be present in one packaging 128, as shown on the right.

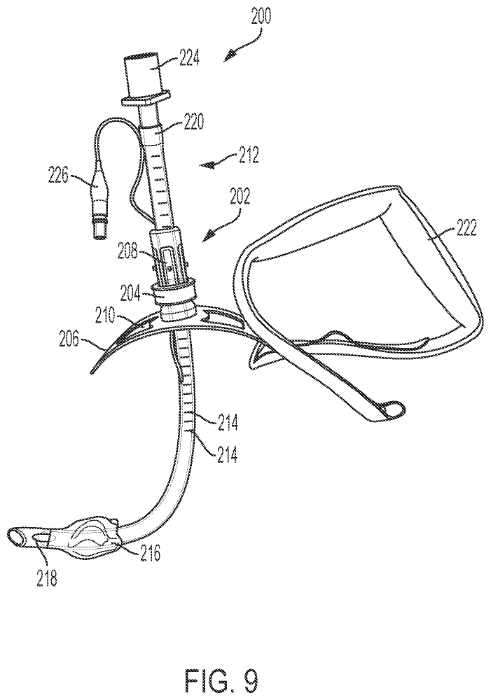

Reference is made to FIG. 9, which shows the endotracheal safety device 200 according to a non-limiting embodiment or aspects. The disclosures of the endotracheal safety device 100 and the disclosures of the endotracheal safety device may be used interchangeably as appropriate. The endotracheal safety device 200 may include an endotracheal sleeve assembly 202, an endotracheal tube 212, and an adapter 224. The endotracheal sleeve assembly 202 may include a flange 206 and a strap 222. The flange 206 may define one or more holes 210. The endotracheal sleeve assembly 202 may include a clamping assembly having a coupler 204 and one or more slits 208. The endotracheal tube 212 may define a proximal end 220 and a distal end 218. The endotracheal tube 212 may include a cuff 216 and a cuff inflation port 226.

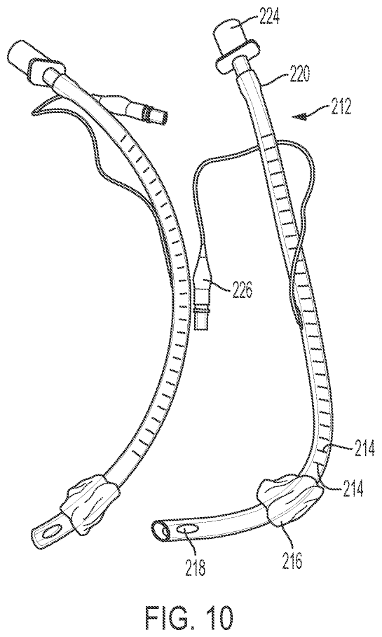

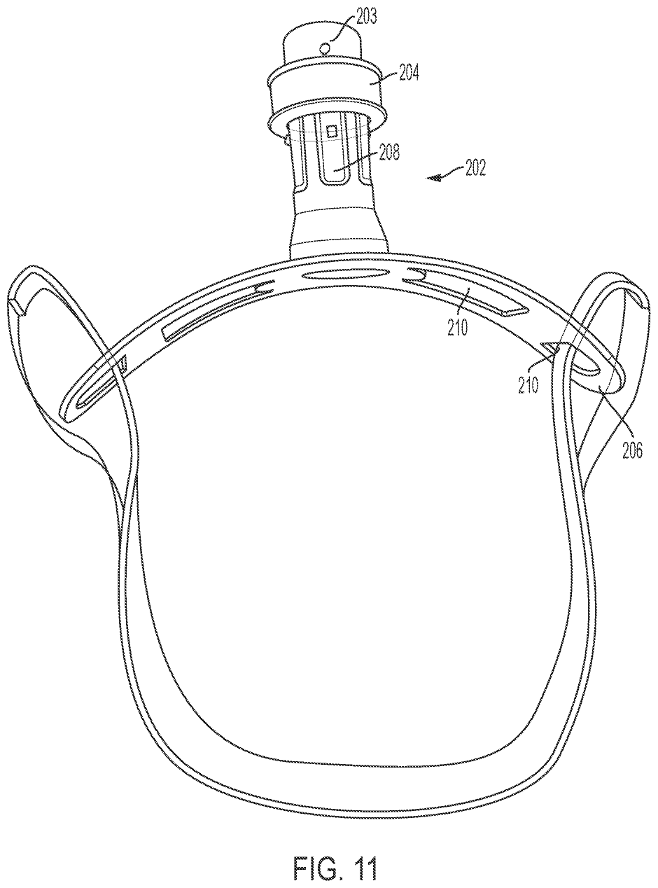

With reference to FIG. 10, the endotracheal tube 212 may have a proximal end 220 that is configured to receive a distal end of the adapter 224. Alternatively, the distal end of the adapter 224 may be configured to receive the proximal end 220 of the endotracheal tube 212. The distal end of the adapter 224 and the proximal end 220 of the endotracheal tube 212 may have corresponding structure that will fasten the adapter 224 and the endotracheal tube 212 in place. For example, the proximal end 220 of the endotracheal tube 212 and the distal end of the adapter 224 may have threads, as to provide a screw fit. The endotracheal tube 212 may further include markings 214 that may indicate how far the endotracheal tube 212 has been inserted into a patient. Additionally, the markings 214 may indicate where the endotracheal sleeve assembly 202 is placed with respect to the endotracheal tube 212. The endotracheal tube 212 may include a cuff 216 and a cuff inflation port 226. The cuff inflation port 226 and the cuff 216 may be in fluid communication such that air introduced to the cuff inflation port 226 may inflate the cuff 216. The distal side of the endotracheal tube 212 may be bent at a substantially 90 degree angle, as shown on the right compared to the non-bent endotracheal tube on the left. For example, the distal side of the endotracheal tube 212 may be bent at an angle between 60 degrees and 90 degrees, 65 degrees and 85 degrees, and 70 degrees and 80 degrees. Such bend in the endotracheal tube 212 allows for easier placement of the endotracheal tube 212 into the trachea of a patient without using an intubating stylet. The bent endotracheal tube 212 may be straightened out once the endotracheal tube 212 is inserted, with the cuff 216 inflates, and the endotracheal tube 212 warms up to the patient's body temperature. The endotracheal tube 212 may define a distal end 218 that is beveled, as shown on the right. The distal end 218 may define an opening, as shown on the left. The opening may coincide with the distal end 218, or may be defined on the tracheal tube 212 a predetermined distance away from the distal end 218, for example, between 0.0 to 1.5 inch, 0.0 to 1.0 inch, and 0.0 to 0.5 inch away from the distal end 218 of the endotracheal tube 212. The opening may be of any suitable shape, such as an oval. The opening may be of any suitable size, for example, between 0.0 inch to 1 inch and 0.25 to 0.75 inch in width and length.

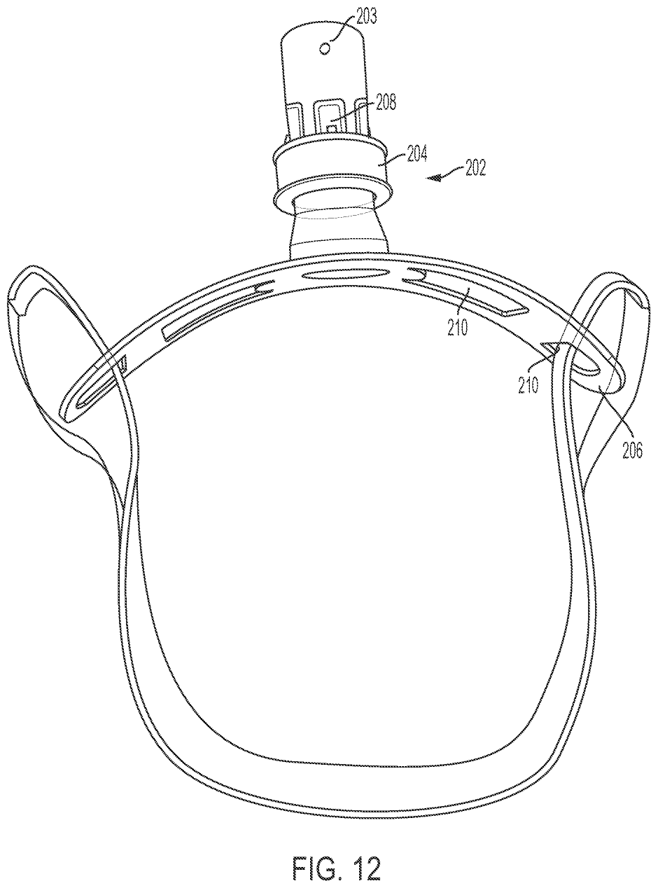

With reference to FIGS. 11-12, the endotracheal sleeve assembly 202 may include a flange 206 defining at least one hole 210, a strap 222, a coupler 204, one or more slits 208, and at least one stop 203 at the proximal end of the endotracheal sleeve assembly 202. FIG. 11 shows the endotracheal sleeve assembly 202 with the coupler 204 separate from the endotracheal sleeve assembly 202. The flange 206 may have a may have a concave shape so as to ergonomically fit over a patient's mouth. FIG. 11 shows the coupler 204 in its first position where the coupler 204 is mounted on the endotracheal sleeve assembly 202 such that the coupler 204 is not exerting any compressive forces on one or more slits 208. The one or more stops 203 prevents the coupler 204 from being disconnected from the endotracheal sleeve assembly 202. The one or more stops 203 may be a flange, an obstruction, or any other suitable structure that prevents the coupler 203 from being removed from the endotracheal sleeve assembly 202. FIG. 12 shows the coupler 204 in its second position where the coupler 204 is mounted on the endotracheal sleeve assembly 204 such that the coupler 204 is placed over one or more slits 208. Then one or more slits 208 may be pressed against the endotracheal tube 212 when the coupler 204 is in its second position. The coupler 204 may be made of any suitable rigid material such that the coupler 204 is configured to apply compressive forces on the one or more slits 208. Such compressive force applied on the one or more slits 208 in turn applies a compressive force onto the endotracheal tube 212. This allows the endotracheal sleeve assembly 202 to be fixed in position in relation to the endotracheal tube 212. The strap 222 may be attached to the flange 206 and/or the at least one hole 210 of the flange. The strap 222 may be used to further secure the endotracheal safety device 200 in place after the endotracheal tube 212 has been inserted into a patient.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure that are known or customarily practiced in the art to which this disclosure pertains and which fall within the limits of the appended claims.

The invention claimed is:

1. An endotracheal safety device comprising:
an endotracheal tube having a proximal end and a distal end, the endotracheal tube defining a lumen between the proximal end and the distal end;
an endotracheal sleeve assembly having a proximal end and a distal end, the endotracheal sleeve assembly defining a lumen between the proximal end and the distal end, the endotracheal sleeve assembly configured to be movable over the endotracheal tube; and
an adapter having a proximal end and a distal end, the distal end of the adapter configured to be received by the proximal end of the endotracheal sleeve assembly.

2. The device of claim 1, wherein a lower portion of the endotracheal tube is bent.

3. The device of claim 1 further comprising a cuff, wherein the cuff is secured on an outer surface of the endotracheal tube at a lower portion of the endotracheal tube, wherein the cuff is inflatable.

4. The device of claim 1, wherein the distal end of the endotracheal tube is tapered.

5. The device of claim 1, wherein the proximal end of the endotracheal tube and the distal end of the adapter are rotationally secured.

6. The device of claim 1, wherein the endotracheal tube has at least one marking on the outer surface.

7. The device of claim 1, wherein the endotracheal sleeve assembly comprises a clamp, wherein the clamp is configured to secure the endotracheal sleeve assembly in place such that the endotracheal sleeve assembly is not movable with respect to the endotracheal tube.

8. The device of claim 1, wherein the endotracheal sleeve assembly further comprises a flange, wherein the flange defines at least one hole.

9. The device of claim 8, wherein the endotracheal sleeve assembly further comprises a strap, wherein the strap is attached to the flange.

10. The device of claim 1 further comprising a cuff inflation port, wherein the cuff inflation port is in fluid communication with the cuff such that the cuff may be inflated by introducing air to the cuff inflation port.

11. The device of claim 1, wherein the endotracheal sleeve assembly comprises at least one slit and a coupler, wherein when the coupler has a first position and a second position over an outer surface of the sleeve assembly, wherein the endotracheal sleeve assembly is configured to move with respect to the endotracheal tube when the coupler is in its first position, and wherein the endotracheal sleeve assembly is secured to the endotracheal tube when the coupler is in the second position.

12. An endotracheal sleeve assembly comprising a proximal end and a distal end, the endotracheal sleeve assembly defining a lumen between the proximal end and the distal end, the endotracheal sleeve assembly configured to be movable over the endotracheal tube.

13. The endotracheal sleeve of claim 12, wherein the endotracheal sleeve assembly comprises a clamp, wherein the clamp is configured to secure the sleeve assembly in place such that the sleeve assembly is not movable with respect to an endotracheal tube, and wherein the endotracheal sleeve assembly comprises at least one stop.

14. The endotracheal sleeve of claim 12, wherein the endotracheal sleeve assembly further comprises a flange, wherein the flange defines at least one hole.

15. The endotracheal sleeve of claim 14, wherein the endotracheal sleeve assembly further comprises a strap, wherein the strap is attached to the sleeve.

16. The endotracheal sleeve of claim 12, wherein the endotracheal sleeve assembly comprises at least one slit and a coupler, wherein when the coupler has a first position and a second position over an outer surface of the endotracheal sleeve assembly, wherein the endotracheal sleeve assembly is configured to move with respect to an endotracheal tube when the coupler is in its first position, and wherein the endotracheal sleeve assembly is secured to the endotracheal tube when the coupler is in the second position.

17. A method of introducing an endotracheal tube into a patient comprising:
inserting a distal end of the endotracheal tube into the patient;
sliding an endotracheal sleeve assembly over the endotracheal tube to a desired length of the endotracheal tube; and
fastening the endotracheal sleeve assembly with respect to the endotracheal tube such that the endotracheal sleeve assembly does not slide with respect to the endotracheal tube.

18. The method of claim 17 further comprising:
providing a strap;
attaching the strap to a flange of the endotracheal sleeve assembly;
wrapping the strap around the patient's head; and
securing the strap to an opposing side of the flange.

19. The method of claim 17 further comprising inflating a cuff of the endotracheal tube by introducing air to a cuff inflation port of the endotracheal tube and straightening the endotracheal tube by exposing the endotracheal tube to the patient's body temperature.

20. The method of claim 17 further comprising fastening a distal end of an adapter to a proximal end of the endotracheal tube.
